SEQUENCE LISTING (2 sequences)

SEQ ID NO: 1 - protein, 48 aa, Homo sapiens
VLSVFCLHKSSCTVAEIYLFLMLVSIDRYLALVKTMSMVYVIVGKRFR

SEQ ID NO: 2 - protein, 59 aa, Homo sapiens
ENIFVLSVFCLHKSSCTVAEIYLEIQTERRATVLPLVYVIVGKRFRKKSWEVYQGVCQK

(19) United States
(12) Patent Application Publication
(10) Pub. No.: US 2016/0220464 A1
(43) Pub. Date: Aug. 4, 2016

(54) NONPEPTIDE BK B2 RECEPTOR AGONISTS FOR HAIR GROWTH

(71) Applicants: Junjie YANG, Wuhan (CN); George LI, Wuhan (CN); Xuefei LI, Wuhan (CN); WUHAN OPTICS VALLEY BRIDGEBIOMED INTERNATIONAL CORPORATION, Wuhan (CN)

(72) Inventors: JUNJIE YANG, Wuhan (CN); GEORGE LI, Wuhan (CN); XUEFEI LI, Wuhan (CN)

(21) Appl. No.: 14/916,971

(22) PCT Filed: Sep. 3, 2014
(86) PCT No.: PCT/US14/53931

§ 371 Date: Mar. 4, 2016

(30) Foreign Application Priority Data
Sep. 6, 2013 (CN) 201310405099.9

(51) Int. Cl. A61K 8/49; G06F 19/18; G06F 19/16; A61Q 7/00; A61K 8/34

(52) U.S. Cl. CPC A61K 8/4946; A61Q 7/00; A61K 8/345; G06F 19/16; G06F 19/18; A61K 2800/74

(57) ABSTRACT

The present invention provides compositions comprising a bradykinin receptor B2 agonist and methods using such compositions to increase/promote hair growth or reduce/delay hair loss.

### FIELD

The invention relates in general to the cosmetic and pharmaceutical fields, and more particularly to compositions and methods for increasing/promoting hair growth or reducing/delaying hair loss.

### BACKGROUND

Bradykinin (BK) receptors belong to the G-protein-coupled receptors (GPCRs). Two different subtypes BK receptors designated B1 and B2 have been identified. B2 receptors are constitutively expressed in a variety of healthy cells and they form a complex with angiotensin converting enzyme (ACE), and thus thought to play a role in cross-talk between the renin-angiotensin system (RAS) and the kinin-kallikrein system (KKS). B1 receptors are constitutively expressed at much lower level, but upregulated by tissue injury, inflammation and cytokines, and are presumed to play a role in chronic pain. B2 receptors are related to a burst of prostaglandin (PG) release, whereas B1 receptors mediate a delayed prostaglandin response. BK agonists that bind to B2 receptors can activate PhospholipaseA2, convert phospholipids to arachidonic acid, which is converted to PGs by Cyclooxygenase. It has also been shown that BK B2 receptors are stimulated in the pathogenesis of inflammation, pain and tissue injury.

Undesired hair loss is a common problem for many men and women. There have been continuous efforts in the cosmetic and pharmaceutical industry searching for substances effective in reducing/delaying hair loss or increasing/promoting hair growth. BK antagonists inhibiting BK receptor signaling have been indicated in treating hair loss, e.g., see U.S. Pat. No. 6,468,972.

### SUMMARY OF THE INVENTION

In one embodiment, the present invention provides a bradykinin (BK) B2 receptor agonist. The agonist may be 4-(3-2-((2,4-dichloro-3-((2-methoxy-1-(pyridine-2ylmethyl)-1H-benzo[d]imidazol-4-yloxy)methyl)phenyl)(methyl)amino-2-oxoethylamino)-3-oxopropyl)-N-methylbenzamid (YLB-01), 3-(6-acetamidopyridin-3-yl)-N-(2-((2,4-dichloro-3-((2-methoxy-1-(pyridine-2-ylmethyl)-1H-benzo[d]imidazol-4yloxy)methyl)phenyl)(methyl)amino-2-oxoethyl) propanamide (YLB-02), or 4-{2-[({[2,4-Dichloro-3-(2-methoxy-1-pyridin-2-ylmethyl-1H-benzo imidazol-4-yloxymethyl)-phenyl]-methyl-carbamoyl}-methyl)-carbamoyl]-ethyl}-piperazine-1-carboxylic acid methylamide (YLB-03). In one embodiment, the invention provides a topical composition comprising at least a bradykinin (BK) B2 receptor agonist as an active ingredient.

The active ingredient has a concentration of from about 0.01% to about 10% by weight according to certain embodiments of the invention.

The active ingredient has a concentration of from about 0.03% to about 3% by weight according to certain embodiments of the invention.

The active ingredient has a concentration of about 0.03% according to certain embodiments of the invention.

The active ingredient has a concentration of about 0.3% according to certain embodiments of the invention.

The active ingredient has a concentration of about 3% according to certain embodiments of the invention.

The active ingredient is a BK B2 receptor agonist selected from the group consisting of YLB-02, YLB-02 and YLB-03.

The active ingredient has a concentration of from 10 nM to 2,000 μM according to certain embodiments of the invention, e.g., from about 10 nM to about 1000 μM, from about 100 nM to about 1,000 μM, from about 100 nM to about 500 μM, from about 100 nM to about 250 μM, from about 100 nM to about 100 μM, from about 100 nM to about 50 μM, from about 100 nM to about 10 μM.

The active ingredient is a non-peptide compound according to certain embodiments of the invention.

The active ingredient, YLB-01, is 4-(3-2-((2,4-dichloro-3-((2-methoxy-1-(pyridine-2ylmethyl)-1H-benzo[d]imidazol-4-yloxy)methyl)phenyl)(methyl)amino-2-oxoethylamino)-3-oxopropyl)-N-methylbenzamid according to certain embodiments of the invention.

The active ingredient, YLB-02, is 3-(6-acetamidopyridin-3-yl)-N-(2-((2,4-dichloro-3-((2-methoxy-1-(pyridine-2-ylmethyl)-1H-benzo[d]imidazol-4yloxy)methyl)phenyl)(methyl)amino-2-oxoethyl) propanamide according to certain embodiments of the invention.

The active ingredient, YLB-03, is 4-{2-[({[2,4-Dichloro-3-(2-methoxy-1-pyridin-2-ylmethyl-1H-benzoimidazol-4-yloxymethyl)-phenyl]-methyl-carbamoyl}-methyl)-carbamoyl]-ethyl}-piperazine-1-carboxylic acid methylamide according to certain embodiments of the invention.

The topical composition according to certain embodiments of the invention may further comprise at least a pharmaceutical carrier selected from the group consisting of transdermal permeation enhancer, transdermal absorption promoting agent, water, solvent, preservative, surfactant, and a pH balancer.

The transdermal permeation enhancer is propylene glycol, Azone, or a combination thereof according to certain embodiments of the invention.

The propylene glycol or Azone has a concentration of 2% by weight according to certain embodiments of the invention.

The topical composition according to certain embodiments of the invention may comprise alcohol and/or a PBS solution.

The PBS solution has a pH at from about 6.5 to about 7.8 according to certain embodiments of the invention.

The topical composition according to certain embodiments of the invention is in the form of a gel, liniment, cream or ointment.

In one embodiment, the present invention provides use of a topical composition as described in the preparation of a medicament for reducing or delaying hair loss.

The hair loss is caused by androgenetic alopecia, or seborrheic alopecia according to certain embodiments of the invention.

In one embodiment, the present invention provides use of a topical composition as described in the preparation of a medicament for increasing or promoting hair growth.

The hair growth is eye brow or eye lash growth according to certain embodiments of the invention.

The hair loss or growth is related to PG E/F2a release according to certain embodiments of the invention.

In one embodiment, the present invention provides a method of reducing or delaying hair loss in a subject comprising administering a topical composition described to the subject in an amount effective to reduce or delay hair loss.

The hair loss is caused by androgenetic alopecia, or seborrheic alopecia according to certain embodiments of the invention.

In one embodiment, the present invention provides a method of increasing or promoting hair growth in a subject, comprising administering a topical composition described to the subject in an amount effective to increase or promote hair growth.

The hair growth is eye brow or eye lash growth according to certain embodiments of the invention.

The hair loss or growth is related to PG E/F2a release according to certain embodiments of the invention.

The daily therapeutic dose of the active ingredient administered is from about 0.01 mg to about 500 mg according to certain embodiments of the invention.

The daily therapeutic dose of the active ingredient administered is from about 0.1 mg to about 300 mg according to certain embodiments of the invention.

The daily therapeutic dose of the active ingredient administered is from about 1 mg to about 250 mg according to certain embodiments of the invention.

In one embodiment, the present invention provides a kit comprising the topical composition as described.

In one embodiment, the present invention provides a computer modeling structure identifying a series of BK B2 receptor agonist binding sites of within BK B2 receptor.

The binding sites are presented in 2-dimentions or 3-dimentions according to certain embodiments of the invention.

The binding sites are identified using molecular modeling according to certain embodiments of the invention.

The molecular modeling is based on one or more BK B2 receptor agonists selected from the group consisting of YLB-01, YLB-02, and YLB-03 according to certain embodiments of the invention.

The residues found at YLB-01 binding pocket border contain one or more selected from the group consisting of G205, I219, Y322, E204, T314, W5, and R297 according to certain embodiments of the invention.

The residues found at YLB-02 binding pocket border contain one or more selected from the group consisting of contain one or more selected from the group consisting of ARG338, TYR332, GLU93, and ARG167 according to certain embodiments of the invention.

The residues found at YLB-03 binding pocket border contain one or more selected from the group consisting of VAL91, GLN352, ARG338, and TYR347 according to certain embodiments of the invention.

The YLB-01 binding site contains one or more residues selected from the group consisting of T95, R55, and R338 according to certain embodiments of the invention.

The YLB-02 binding site contains one or more residues selected from the group consisting of E93, Y332, and R338 according to certain embodiments of the invention.

The YLB-03 binding site contains one or more residues selected from the group consisting of V91, R338, Y347, and Q352 according to certain embodiments of the invention.

### BRIEF DESCRIPTION OF THE DRAWINGS

FIG. 1 is a simplified, schematic illustration showing new hairs grown after YLB-01 suspension treatment.

FIG. 2 is a simplified, schematic illustration showing enhanced hair growth after non-peptide B2 agonist treatment.

FIG. 3 is a simplified, schematic illustration showing PFSC code of YLB-01 binding site.

FIG. 4 is a simplified, schematic illustration showing PFSC code of YLB-02 binding site.

FIG. 5 is a simplified, schematic illustration showing PFSC code of YLB-03 binding site.

FIG. 6 is a simplified, schematic illustration showing YLB-01 assay by HPLC.

FIG. 7 illustrates the pharmacophore features, intracellular view of the activated B2R and of three non-peptide B2 receptor agonists.

### DETAILED DESCRIPTION

The present invention provides compositions and methods for effective treatment of hair loss and promotion of hair growth. The inventors of the present invention discovered that bradykinin (BK) B2 receptor agonists could stimulate BK receptor B2 and therefore trigger secretion of endogenous prostaglandins (PG) F-2a; local microenvironment favoring hair growth could be improved by PG receptor pathway, which makes it a novel drug for hair growth promotion.

This invention further provides a method of developing novel drug against hair loss through regulating the amount and proportion of PG and PG subtypes within local microenvironment.

The drug could be directly applied by topical application to the treatment sites, and take effect locally, thus reduce the side effect of systemic drug application. The present invention relates to a series of compounds and pharmaceutical compositions comprising one or more of these compounds and methods of administering them for the treatment of PG D/PG F2a ratio related hair loss by increasing PG-F 2a release.

BK B2 receptor agonists may be used in the compositions and methods according to some embodiments of the present invention.

All of agonists activated the BK B2 receptor, working as an endogenous ligand bradykinin to increase the release of PGs. This effect was blocked by the B2R antagonist HOE 140.

Prostaglandins (PG) are a group of lipid compounds and have important functions in the animal body. Prostaglandin was first isolated from seminal fluid and it was believed to be part of the prostatic secretions, therefore it was named prostaglandins. Now it has been proved that prostaglandins are mainly produced by the seminal vesicles, and could be produced by cells from most tissues and organs.

The prostaglandins are produced in vivo from arachidonic acids, and every prostaglandin contains 20 carbon atoms, including a 5-carbon ring. There're different types of prostaglandins, i.e. A, B, C, D, E, F, G, H, I, since their structures differ. Different types of prostaglandins have different functions, e.g. Prostaglandin E could diastole bronchial smooth muscle, thus decrease airway resistance; while Prostaglandin F has the opposite functionalities. The half-life of prostaglandins are very short (usually from 1-2 minutes), and all the prostaglandins except Prostaglandin I degrade quickly through liver and lung. Unlike typical hormones affecting a discrete site through the circulatory system, prostaglandins are produced and secreted locally, and modulate the activities at their sites of secretion.

Previous research indicates that prostaglandin F-2a promotes hair growth, while prostaglandin D2 inhibits hair growth; which suggests prostaglandins are crucial in local endocrine modulation as well as hair growth.

PGE2 is known to act synergistically with PGF2alpha, and hence the influence of PGE2 was also examined. PGE2 did not induce distinct telogen-to-anagen conversion, but showed moderate growth stimulatory effects on early anagen hair follicles.

Kinins, Bradykinin (BK) agonists, must first bind to cell surface receptors in order to exert their biological effects. BK receptors belong to G-protein-coupled receptors (GPCRs). There are at least two different subtypes, B1 and B2 receptors. It was reported that the two receptor subtypes are linked to different signal transduction mechanisms. BK agonists that can bind to B2 receptor activate PhospholipaseA2. This enzyme converts phospholipids to arachidonic acid. Cyclooxygenase 1&2 in turn catalyze the formation of PGs from this fatty acid.

Direct activation of the kinin B2 receptor by pharmacological agonists might provide higher therapeutic benefit than existing kinin-potentiating drugs. Local application of B2 receptor agonists stimulates the endogenous prostaglandin secretion.

Latanoprost, analogues of prostaglandin F2alpha (PGF2alpha), used clinically in the treatment of glaucoma, induces growth of lashes and ancillary hairs around the eyelids. The increased length of lashes is consistent with the ability of latanoprost to prolong the anagen phase of the hair cycle. Correlation with laboratory studies suggests that initiation and completion of latanoprost hair growth effects occur very early in anagen and the likely target is the dermal papilla.

Three new B2 receptor agonists (YLB-01, YLB-02, YLB-03) were evaluated in this study. YLB-01 is 4-(3-2-((2,4-dichloro-3-((2-methoxy-1-(pyridine-2ylmethyl)-1H-benzo[d]imidazol-4-yloxy)methyl)phenyl)(methyl)amino-2-oxoethylamino)-3-oxopropyl)-N-methylbenzamid. YLB-02 is 3-(6-acetamidopyridin-3-yl)-N-(2-((2,4-dichloro-3-((2-methoxy-1-(pyridine-2-ylmethyl)-1H-benzo[d]imidazol-4yloxy)methyl)phenyl)(methyl)amino-2-oxoethyl) propanamide. YLB-03 is 4-{2-[({[2,4-Dichloro-3-(2-methoxy-1-pyridin-2-ylmethyl-1H-benzo imidazol-4-yloxymethyl)-phenyl]-methyl-carbamoyl}-methyl)-carbamoyl]-ethyl}-piperazine-1-carboxylic acid methylamide. Our results showed that prostaglandin F-2a and PGE2 could be induced by these three compounds but PGD2 was not detectable. Meanwhile YLB-01 is effective in reducing/delaying hair loss or increasing/promoting hair growth. Since local application of B2 receptor agonists stimulates the endogenous prostaglandin secretion, local microenvironment favoring hair growth could be improved by PG receptor pathway, which makes a novel drug possible for hair growth promotion.

As used herein, the term “subject” means an animal, preferably a mammal, and most preferably a human. A subject may be a patient having an undesired hair loss problem or a need for hair growth as discussed herein.

As used herein, the term “active ingredient” refers to an active substance that has activity or effect of increasing/promoting hair growth and/or decreasing/delaying hair loss. The term “active ingredient” of the present invention includes a physiologically acceptable salt form of the active ingredient. The term also includes a prodrug which releases an active substance in vivo when such prodrug is administered to a subject. The term is used interchangeably with the term “drug” according to some embodiments of the present invention.

As used herein, the term “physiologically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio.

The term “increasing” or “promoting” refers to a positive change in the amount of hair growth. The positive change may be about 5%, about 10%, about 25%, about 50%, about 75%, about 90%, about 100% or more when compared to a control amount hair growth. For example, a composition of the present invention may increase or promote hair growth in a subject by about 5%, about 10%, about 25%, about 50%, about 75%, about 90%, about 100% or more as compared to the amount of hair growth without the application of the composition of the present invention.

As used herein, the term “reducing” or “delaying” refers to a negative change in the amount of hair loss or progression of hair loss. The negative change may be about 5%, about 10%, about 25%, about 50%, about 75%, about 90%, about 100% when compared to a control amount or progression of hair loss. For example, a composition of the present invention may reduce hair loss in a subject by about 5%, about 10%, about 25%, about 50%, about 75%, about 90%, about 100% as compared to the amount of hair loss without the application of the composition of the present invention.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 30%, preferably 20%, more preferably 10%.

As used herein, the term “comprises” means “includes, but is not limited to.”

The term “physiologically acceptable carrier” is used herein to refer to a material that is compatible with a recipient subject, preferably a mammal, more preferably a human, and is suitable for delivering an active ingredient to the target site without terminating the activity of the active ingredient. The toxicity or adverse effects, if any, associated with the carrier preferably are commensurate with a reasonable risk/benefit ratio for the intended use of the active agent.

The term “carrier” is used interchangeably herein, and includes any and all solvents, diluents, and other liquid vehicles, dispersion or suspension aids, surface active agents, isotonic agents, thickening or emulsifying agents, preservatives, solid binders, lubricants and the like, as suited to the particular dosage form desired. Remington: The Science and Practice of Pharmacy, 20th Ed., ed. A. Gennaro, Lippincott Williams & Wilkins, 2000 discloses various carriers used in formulating physiologically acceptable compositions and known techniques for the preparation thereof.

The term “transdermal permeation enhancer” means a natural or synthetic molecule which facilitates the absorption of a given active agent or combination of active agents through the skin, e.g., as a portal for the administration of drugs by topical application.

A transdermal permeation enhancer may result in an increased aggregate transdermal delivery over a period of time (e.g., 3, 6, 9, 12 or 24 hours) by 5%, 10%, 25%, 50%, 100%, 2-folds, 5-folds, or more. Transdermal permeation enhancers of the present invention include, but are not limited to, those with diverse mechanisms of action including those which have the function of improving the solubility and diffusibility of the drug within the multiple polymer and those which improve percutaneous absorption, for example, by changing the ability of the skin to retain moisture, softening the skin, improving the skin's permeability, acting as permeation assistants or hair-follicle openers or changing the state of the skin including the boundary layer.

Permeation enhancers suitable for use with the present invention include, but are by no means limited to, natural or synthetic molecules which facilitate the absorption of an active agent through skin Some examples of permeation enhancers are polyhydric alcohols such as dipropylene glycol, propylene glycol and polyethylene glycol which enhance drug solubility; oils such as olive oil, squalene, and lanolin; fatty ethers such as cetyl ether and oleyl ether; fatty acid esters such as isopropyl myristate which enhance drug diffusibility; urea and urea derivatives such as allantoin which affect the ability of keratin to retain moisture; polar solvents such as dimethyldecylphosphoxide, methyloctyl-sulfoxide, dimethyllaurylamide, dodecylpyrrolidone, isosorbitol, dimethyl-acetonide, dimethylsulfoxide, decylmethyl-sulfoxide, and dimethylformamide which affect keratin permeability; salicylic acid which softens the keratin; amino acids which are permeation assistants; benzyl nicotinate which is a hair follicle opener; and higher molecular weight aliphatic surfactants such as lauryl sulfate salts which change the surface state of the skin and drugs administered. Other agents include oleic and linoleic acids, ascorbic acid, panthenol, butylated hydroxytoluene, tocopherol, tocopheryl acetate, tocopheryl linoleate, propyl oleate, isopropyl palmitate and glyceryl monooleate. The concentration of each enhancer may be between 0.1 to 20%.

In some embodiments of the present invention, the permeation enhancer is propylene glycol or azone or a combination of both.

As with percentages disclosed herein, these percentages may be mass per mass (i.e., by weight). In some embodiments, the ingredients of the topical compositions disclosed herein are provided in amounts ranging from about 1 μg to about 1 g, or higher (e.g., 1 μg-2 μg, 2 μg-5 μg, 5 μg-10 μg, 10 μg-25 μg, 25 μg-100 μg, 100 μg-500 μg, 500 μg-1 mg, 1 mg-5 mg, 5 mg-10 mg, 10 mg-20 mg, 20 mg-30 mg, 30 mg-40 mg, 40 mg-50 mg, 50 mg-60 mg, 60 mg-70 mg, 70 mg-80 mg, 80 mg-90 mg, 90 mg-100 mg, 100 mg-250 mg, 250 mg-500 mg, 500 mg-1 g, or higher, and overlapping ranges thereof). These amounts may be the weight of the ingredient per individual application (or dose), per unit or per container (tube, bottle, jar, etc.). Individual applications may be made hourly, 1-10 times or 2-5 times per day, weekly, or as needed. The concentration of the active ingredient may be from about 1 to about 100 mg per ml, e.g., from about 3 to about 50 mg per ml. The concentration of the active ingredient may be from about 10 nM to about 2,000 μM, e.g., from about 10 nM to about 1000 μM, from about 100 nM to about 1,000 μM, from about 100 nM to about 500 μM, from about 100 nM to about 250 μM, from about 100 nM to about 100 μM, from about 100 nM to about 50 μM, from about 100 nM to about 10 μM.

The active ingredient described herein may be formulated for administration with a pharmaceutical carrier in accordance with known techniques. See, e.g., Remington, The Science And Practice of Pharmacy (9^{th} Ed. 1995), herein incorporated by reference. In the manufacture of a composition according to several embodiments of the invention, the active ingredient (including the physiologically acceptable salts thereof) may be admixed with, inter alia, an acceptable carrier. One or more active ingredients may be incorporated in the compositions of the invention, which may be prepared by any of the well-known techniques of pharmacy comprising admixing the components, optionally including one or more accessory ingredients. Carriers which may be used include petroleum jelly, lanoline, polyethylene glycols, alcohols, transdermal enhancers, and combinations of two or more thereof. In some embodiments, the topical composition is partially or fully incorporated in delivery vehicles such as microspheres or nanoparticles, or is encapsulated (e.g., in liposomes). In some embodiments, a topical composition is pre-impregnated in/on a support structure (e.g., tape, patch, bandage, etc.).

The compositions of the present invention may comprise one or more active ingredients according to some embodiments. In addition, the compositions disclosed herein comprise other additives, such as pH-adjusting additives. In some embodiments, useful pH-adjusting agents include acids, such as citric acid or lactic acid, bases or buffers, such as sodium lactate, sodium acetate, sodium phosphate, sodium citrate, sodium borate, or sodium gluconate.

The compositions may contain antimicrobial preservatives in some embodiments. In several embodiments, antimicrobial preservatives include, but are not limited to, methylparaben, propylparaben, benzyl alcohol, ethylhexylglycerin, potassium sorbate, phenoxyethanol, EDTA, grapefruit seed extract, tea tree oil, sodium benzoate, dehydroacetic acid, and combinations thereof. In some embodiments, anti-fungal preservatives are used alone or in combination with anti-bacterial preservatives.

### EXAMPLES

The present description is further illustrated by the following examples, which should not be construed as limiting in any way. The contents of all cited references (including literature references, issued patents, and published patent applications as cited throughout this application) are hereby expressly incorporated by reference.

### Example 1

We studied non-peptide agonists activating bradykinin (BK) B2 receptors to promote secretion of Prostaglandin followed PG kit instruction. YLB-01, YLB-02 and YLB-03 were selected for the study as described below in Table 1, and BK was used as control. Implantation Two cell lines were transferred into T-75 cell culture flask, and then the instructions of PG kit were strictly followed when cells have reached the appropriate density. Day 1 Cells were seeded in 24 well dishes with minimum medium and allowed to adhere overnight. Day 2 Cells were starved in the serum-free conditioned medium. Day 3 Minimum medium were used, and YLB-01, YLB-02 and YLB-03 were added to the medium, in which the concentration was 0.1 nM, 1 nM, 10 nM, 30 nM, 100 nM, 300 nM, 1 μM, 3 μM and 10 μM, respectively; the cells were incubated at 37° C. for 1 h. The supernatant was collected after incubation for later use. Day 4 24 well dishes were washed and then dyed for 45-90 min, and then the OD value was measured for analysis. Therefore OD values of BK B2 receptor agonists were obtained and the EC₅₀, Eₘₐₓ were calculated as shown in Table 2. The cell strains used in the study were human primary vein endothelial cells (HPVEC) and human aortic smooth muscle cells (HASMC).

TABLE 1
Non peptide BK B2 agonists

| Name | YLB-01 |
|---|---|
| Structure | 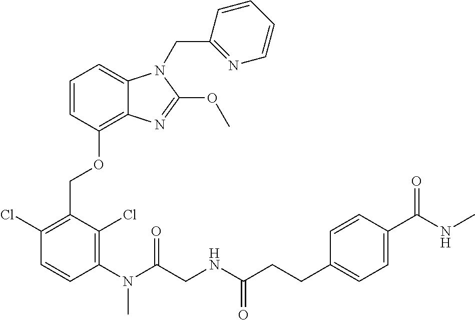 [SMILES: CNC(=O)c1ccc(CCC(=O)NCC(=O)N(C)c2ccc(Cl)c(COc3cccc4c3nc(OC)n4Cc3ccccn3)c2Cl)cc1] |
| Formula | C₃₅H₃₄Cl₂N₆O₅ |
| MW | 689.60 |
| Name | YLB-02 |
| Structure | 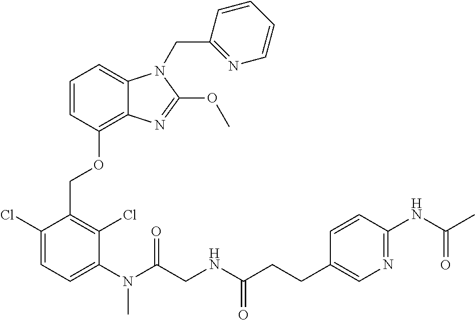 [SMILES: COc1nc2c(OCc3c(Cl)ccc(N(C)C(=O)CNC(=O)CCc4ccc(NC(C)=O)nc4)c3Cl)cccc2n1Cc1ccccn1] |
| Formula | C₃₄H₃₃Cl₂N₇O₅ |
| MW | 690.59 |
| Name | YLB-03 |
| Structure | 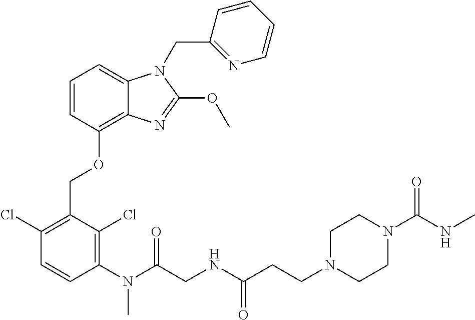 [SMILES: CNC(=O)N1CCN(CCC(=O)NCC(=O)N(C)c2ccc(Cl)c(COc3cccc4c3nc(OC)n4Cc3ccccn3)c2Cl)CC1] |
| Formula | C₃₃H₃₈Cl₂N₈O₅ |
| MW | 697.62 |

TABLE 2
EC₅₀ and Eₘₐₓ of non peptide BK B2 agonists

| | HASMC | | |
|---|---|---|---|
| Drug/cells | EC₅₀ (nM) | Eₘₐₓ (%) | n |
| BK | 7.4 ± 1.4 | 100.6 ± 5.5 | 10 |
| YLB-01 | 24.4 ± 3 | 87.79 ± 4.2 | 16 |
| YLB-02 | 40.65 ± 4.19 | 70.44 ± 5.06 | 13 |
| YLB-03 | 26.23 ± 2.87 | 103.48 ± 9.06 | 12 |

The results above showed that EC₅₀ of BK is the lowest, EC₅₀ of YLB-02 is the highest, more than 5 times EC₅₀ of BK, but Eₘₐₓ is just as 70% BK. Eₘₐₓ of YLB-01 and YLB-03 is 87% and 103% BK, respectively.

### Example 2

In order to detect endogenous prostaglandin, enzyme immunoassay (EIA) was used to determine the amount of prostaglandin and subtypes thereof in the cell supernatant described above. The EIA protocol took 18 hours, and the results were shown in Table 3 below. The results indicated the effect of 10 nM of YLB-01 was roughly equal to 50% of the effect of 1 μM of BK, while the effect of 30 nM of YLB-02 was nearly equal to 40% of the effect of 1 μM of BK. Moreover, the effect of 30 nM of YLB-03 was nearly equal to 55% of the effect of 1 μM of BK; Therefore, the order of decreasing efficiency of the tested non-peptide BK B2 receptor agonists is: YLB-03, YLB-01, and YLB-02. 3 μM doses of these three compounds showed the same results.

TABLE 3
The Relative Amount of PG Secretion by three compounds

| | | Drugs | | |
|---|---|---|---|---|
| Concentration | | YLB-01 | YLB-02 | YLB-03 |
| 10 | μM | 86.48 ± 7.44 | 65.53 ± 5.78 | 103.48 ± 9.06 |
| 3 | μM | 87.79 ± 4.2 | 70.44 ± 5.06 | 95.59 ± 7.21 |
| 1 | μM | 86.98 ± 3.76 | 66.94 ± 8.11 | 85.04 ± 4.83 |
| 300 | nM | 83.83 ± 6.45 | 67.35 ± 7.02 | 86.23 ± 8.51 |
| 100 | nM | 73.43 ± 2.7 | 65.64 ± 8.10 | 70.95 ± 4.57 |
| 30 | nM | 53.71 ± 4.56 | 41.08 ± 6.97 | 55.68 ± 4.09 |
| 10 | nM | 46.13 ± 3.11 | 26.40 ± 6.06 | 47.33 ± 4.22 |
| 1 | nM | 7.69 ± 2.19 | 15.21 ± 3.36 | 10.31 ± 2.30 |
| 0.1 | nM | 0.00 ± 1.54 | 0 ± 1.32 | 0.44 ± 1.22 |

PG secretion amount released by 1 μM BK was set as 100%

The detailed protocol for the elucidation of inhibited PG secretion by B2 receptor inhibitor HOE-140 was shown as follows. Two different cell lines were transferred into T-75 cell culture flask, and then the instructions of PG kit were strictly followed when cells have reached the appropriate density. Day 1. Cells were seeded in 24 well dishes with minimum medium and allowed to adhere overnight. Day 2. Cells were starved in the serum-free conditioned medium. Day 3. Minimum medium were used, and 0.1 nM, 1 nM, 10 nM, 30 nM, 100 nM, 300 nM, 1 μM, 3 μM and 10 μM of three compounds were added to the medium, respectively, and 1 μM of HOE-140 was added at the same time. Cells were incubated at 37° C. for 1 h. The supernatant was collected after incubation for later use. Day 4. 24 well dishes were washed and then dyed for 45-90 min, and then the quantitative analysis was carried out. Therefore OD values (420 nm) of BK B2 receptor agonists were obtained and relative PG amounts were calculated.

The results of quantitative study shown below in Table 4 indicate that 1 μM HOE-140 could block more than 90% of PG synthesis caused under 10 μM of these three compounds. However, if these compound doses were higher than 10 μM, 1 μM HOE-140 could not block the effects of these compounds on PG release. Therefore we could conclude these compounds influence PG secretion via B2 receptor since HOE-140 acts as B2 receptor antagonist.

TABLE 4
Relative PG Secretion Amount with 1 μM HOE-140

| | | Drugs | | |
|---|---|---|---|---|
| | | YLB-01 + | | YLB-03 + |
| | | HOE-140 | YLB-02 + HOE-140 | HOE-140 |
| Concentration | | 1 μM | 1 μM | 1 μM |
| 10 | μM | 40.32 ± 3.27 | 10.1 ± 4.43 | 26.08 ± 7.56 |
| 3 | μM | 6.98 ± 1.6 | 4.25 ± 2.06 | 7.33 ± 4.12 |
| 1 | μM | 3.66 ± 2.11 | 2.87 ± 3.04 | 5.14 ± 2.67 |
| 0.1 | nM | 1.54 ± 2.53 | 3.65 ± 2.77 | 0.54 ± 3.59 |
| 1 | nM | 4.21 ± 3.74 | 0.21 ± 4.42 | 2.65 ± 3.81 |
| 10 | nM | 4.17 ± 3.21 | 5.60 ± 3.43 | 5.64 ± 4.05 |
| 30 | nM | 2.86 ± 2.35 | 3.33 ± 1.88 | 4.90 ± 4.83 |
| 100 | nM | 3.44 ± 3.77 | 0.43 ± 4.16 | 3.97 ± 2.75 |
| 300 | nM | 0.45 ± 2.86 | 1.05 ± 2.39 | 0.48 ± 3.50 |

PG secretion amount released by 1 μM BK was set as 100%

### Example 3

In order to study of prostaglandin subtypes secretion triggered by activation of B2 receptor by these three compounds, human primary vein endothelial cells (HPVEC) and human aortic smooth muscle cells (HASMC) were used for PG secretion, following the same protocol above; 10 nM of these compounds, YLB-1, YLB-2, YLB-3 was added. Then EIA was employed to determine the amount of PG subtypes, and the results shown below in Table 5 indicated that about 30% of PG secretion induced by these B2 agonists was Prostaglandin E2, while roughly 6% was Prostaglandin F-2a and there was no Prostaglandin D detected. These percentage values above were all calibrated with respect to bradykinin. However bradykinin failed to penetrate through skin, and moreover, it has obvious adverse effect and toxicity, hence bradykinin has yet become clinical medication.

TABLE 5
The PG Subtype Percentage

| Drugs | Cell lines | PGE2 (%) | PGF-2a (%) | PGD2 (%) |
|---|---|---|---|---|
| YLB-01 | HASMC | 34.64 ± 12.27 | 5.68 ± 2.08 | undetectable |
| | HPVEC | 30.24 ± 5.40 | 6.29 ± 1.01 | undetectable |
| YLB-02 | HASMC | 26.33 ± 6.78 | 7.1 ± 3.41 | undetectable |
| | HPVEC | 34.15 ± 9.54 | undetectable | undetectable |
| YLB-03 | HASMC | 27.83 ± 10.66 | 8.43 ± 3.57 | undetectable |
| | HPVEC | 31.04 ± 8.49 | undetectable | undetectable |

PG secretion amount released by 1 μM BK was set as 100%
*HPVEC: Human Primary Vein Endothelial Cells;
*HASMC: Human Aortic Smooth Muscle Cells

### Example 4

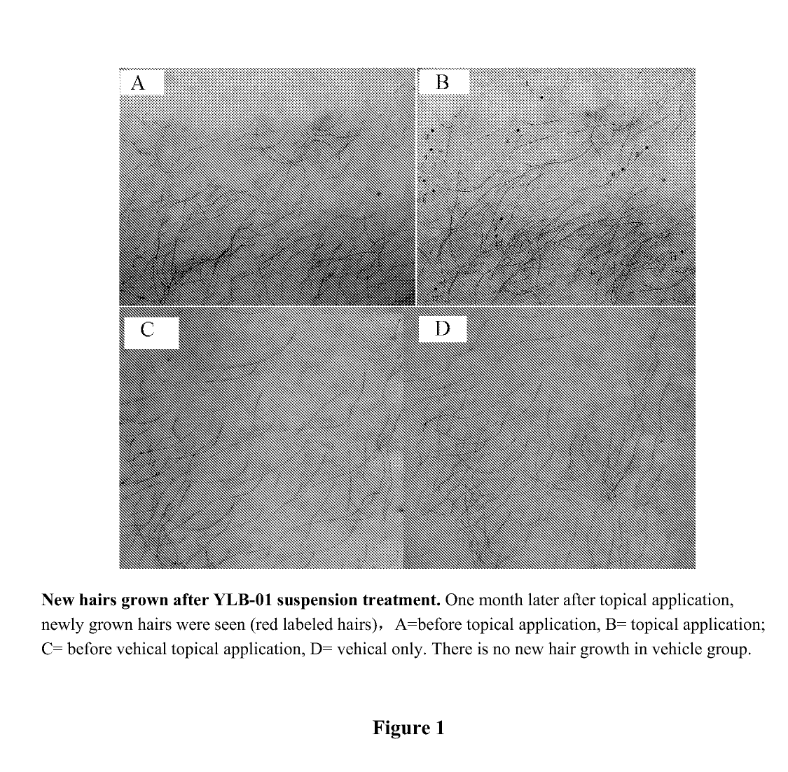

The B2 receptor agonist (YLB-01) suspension with 0.3% concentration was obtained for clinical use as described above. The suspension was applied to human lower leg twice a day, for 3 months; hair growth was observed. The treated areas were observed daily to monitor hair growth and side effects, e.g. swelling, pruritus, pain, etc. And the treated parts were photographed weekly. As shown in FIG. 1, the photos suggested that obvious improvement appeared after a month. And it could be seen that tiny new hairs grew out in the parts used to be bald, as shown in FIG. 1(B). However there is no new hair growth in control group as shown in FIG. 1(D).

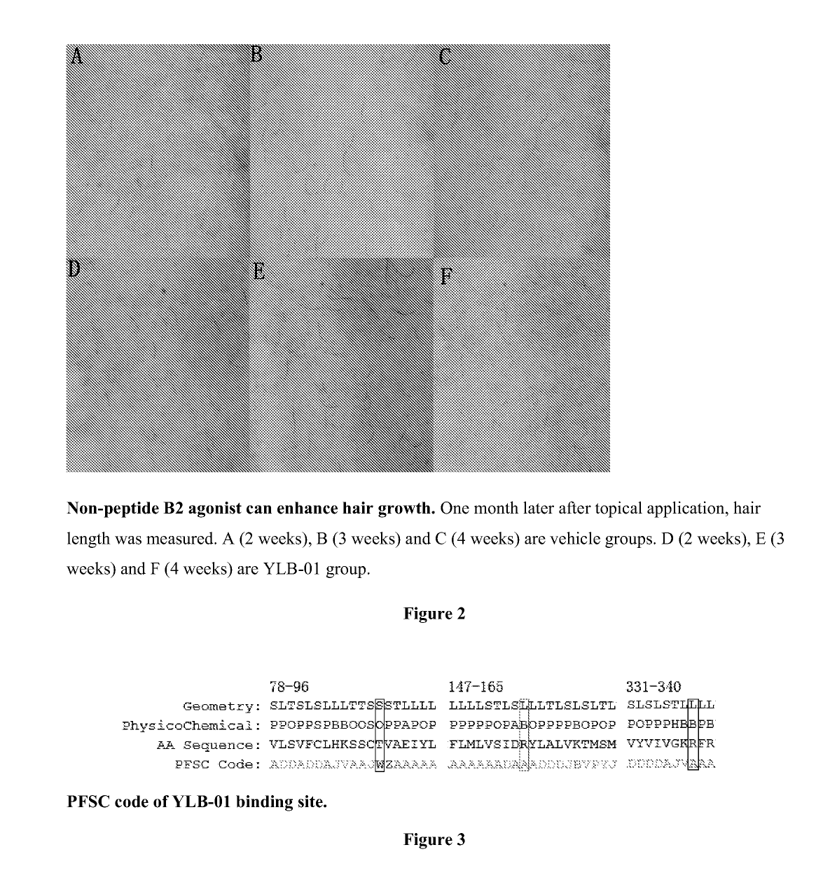

The B2 receptor agonist (YLB-01) suspension with 0.3% concentration was obtained for clinical use as described above and was applied to human lower leg twice a day, for 3 months; hair growth was observed. The treated areas were observed daily to monitor hair growth and side effects, e.g. swelling, pruritus, pain, etc. And the treated parts were photographed weekly. As shown in FIG. 2, the photos suggested that obvious improvement appeared after 2 weeks. And it could be seen that hairs grew more quickly than those in control group, as shown in FIG. 2(D) and Table 6.

TABLE 6
Effect of non-peptide BK B2 receptor (YLB-01) on hair growth (mm)

| | 0 week | 2 weeks | 3 weeks | 4 weeks |
|---|---|---|---|---|
| Control | 0 | 4.6 ± 0.57 | 6.22 ± 0.65 | 8.92 ± 0.94 |
| YLB-01 | 0 | 4.89 ± 0.38 | 6.82 ± 0.4 | 9.61 ± 0.55 |
| | | P < 0.05 | P < 0.001 | P < 0.01 |

### Example 5

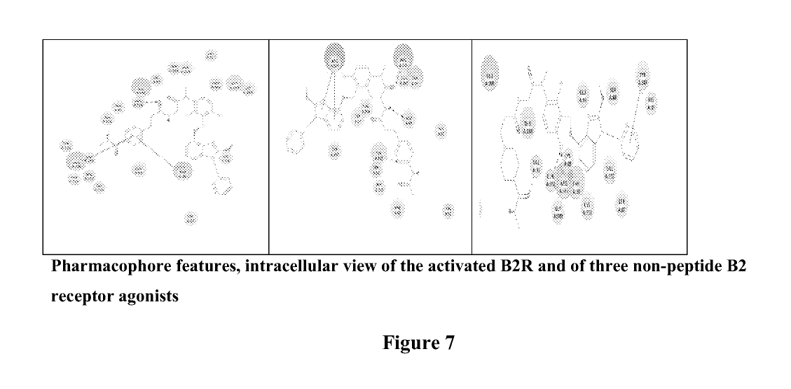

B2 receptor binding was analyzed by homology modeling techniques. B2R belongs to the superfamily of G protein-coupled receptors (GPCRs). Accordingly, we want to carry out a study of the interaction between B2 receptor agonist and B2R. Although computational studies have previously been performed to investigate hormone/ligand-GPCR complex structures at the molecular level, only two crystal GPCR structures have been solved: the ground state rhodopsin (Rh) structure and the inactive form of the β2-adrenergic receptor (β2-ADR). Bradykinin receptors B1 and B2 belong to the class A family of the rhodopsin-like GPCRs. Since the B2R structure is not available, the high-resolution crystal structure of bovine rhodopsin (PDBID: 1HZX) was used as a template for the B2 receptor modeling by using homology modeling techniques. The B2 receptor sequence (entry P30411) was taken from the Swiss-Prot Data Base. An initial 3D homology model was built by using the MACROMOLECULES module from the Discovery Studio 3.5 software package. MD simulation is performed by using CHARMm forcefield. Then the model structure was further evaluated using The Ramachandran Plot and the Verify Protein (Profiles-3D) protocol (FIG. 7).

Structures of YLB-01, YLB-02 and YLB-03 were obtained from Chemdraw 3.0 and CHARMm forcefield was applied. Building a model for the YLB-1,2,3-B2R complex ligand docking: The receptor pocket is conserved within the class A GPCR family. The Input Site Sphere of the receptor pocket is located −0.164482, 6.6825, 22.61, 22.4 respectively. We launch the CDOCKER which is a powerful CHARMm-based docking method to generate highly accurate docked poses. (FIG. 7).

Residues found at YLB-01 binding pocket border are G205, I219, Y322, E204, T314, W5, and R297. Similarly, residues found at YLB-02 binding pocket border are ARG338, TYR332, GLU93, and ARG167. Residues found at YLB-03 binding pocket border are VAL91, GLN352, ARG338, TYR347. Therefore we can conclude that these three drugs were binding into the different pockets (FIG. 7).

Usually the pharmacophore features also include hydrophobicity (Light Blue), aromaticity (Yellow), hydrogen bond acceptor (Green), hydrogen bond donor (Violet), negative ionization (Dark Blue). Each HBA function has two features because of the directionality of hydrogen bond: the position of related heavy atoms and the projection spot, which is the hydrogen bond direction toward the target. Each sphere defines the spatial tolerance of a specific site as shown FIG. 7 row 4.

In the 2D map above, green arrow stands for hydrogen bond. The green dotted line represents the interaction with hydrogen bond of amino acid main chain, the arrow points to the electron donor. Meanwhile the blue dotted arrow represents the interaction with amino acid side chain and points towards electron donor. Green circles represent the residues interacted via Van Der Waals force. Blue hale around residues stands for solvent-accessible interaction, and the radius is proportional to the solvent-accessible surface. The hydrogen bond between ligands and acceptors are represented with green dotted line. The hydrogen atoms are omitted in the map, PI electron interaction is represented with an orange line, and colors differ as amino acids vary as shown FIG. 7 row 5,6.

Meanwhile, we use PFSC code to describe the three-dimensional protein binding site. The numbers represent binding sites on the chain. Blue frames represent hydrogen bond and PI interaction which amino acids involve. The results are shown in FIG. 1 and FIG. 2. Geometry: the amino acid properties illustration, tiny=T, small=S, large=L hardiness=H. Phy-Chem=Chemical-Physical Properties, hydrophobicity=P, acidic=A, basic=B, the Glycine=H, OH═O, carbonyl and amide group O═CNH2=N, SH═S. PFSC Code: Structural assignments from PFSC method, “A” indicating α-helix and “B” β-strand. The PFSC in violet represent secondary structure like carriers.

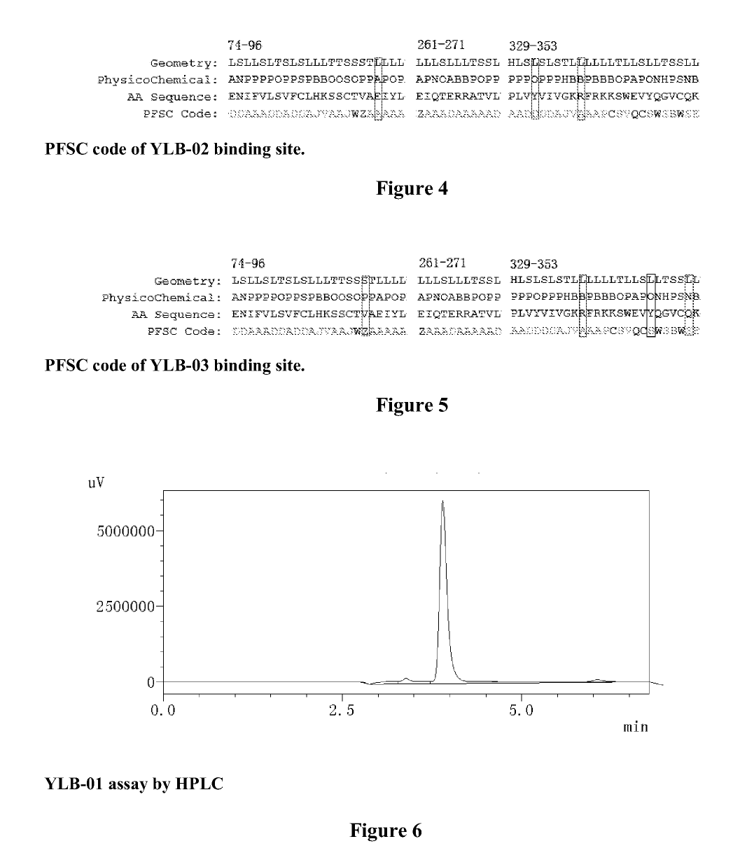

The Probe Binding Site for Drug Discovery (PBSDD) technology is one of applications of Protein Folding Shape Code (PFSC), which provides a power tool to make high-throughput of given protein database revealing drug binding information for drug discovery. We use PFSC code to describe the three-dimensional protein binding sites of YLB-01, YLB-02, YLB-03 as shown in FIG. 3-5, respectively.

According to the number of binding sites, the binding affinity in descending order is: YLB-02, YLB-01, and YLB-03. YLB-02 has stronger binding affinity than YLB-01, and YLB-03 according to the number of binding sites (Table 7 and 8). However, based on release of PGs and their subtypes by these compounds, release strength is not related to affinity. In other words, at given level of affinity, release function of PGs by these compounds is nearly same. Therefore the difference between functional groups determines the affinity of the drugs, which is the molecular basis of our patent claims.

TABLE 7
Number of Pharmacophore Features of B2 receptor agonist

| | | | Hydrogen | Hydrogen | |
|---|---|---|---|---|---|
| | Negative | | bond | bond | |
| Drugs | ionizable | Aromatic | acceptor | donor | Hydrophobic |
| YLB-01 | 0 | 3 | 2 | 0 | 1 |
| YLB-02 | 0 | 3 | 2 | 1 | 1 |
| YLB-03 | 0 | 3 | 0 | 0 | 1 |

TABLE 8
Residues found at binding pocket border

| | Hydrogen bond | Hydrogen bond | |
|---|---|---|---|
| | acceptor | donor | PI interaction |
| YLB-01 | G205, I219, Y322 | E204, T314, Y322 | W5, R297 |
| YLB-02 | ARG338, TYR332 | GLU93 | ARG167 |
| YLB-03 | VAL91, | 0 | TYR347 |
| | GLN352, ARG338 | | |

### Example 6

To explore the optimized dosage form and preparation technology of BK B2 receptor agonist, we selected YLB-01, which has the long-term stability for preservation, as a sample from three agonists (YLB-01, YLB-02, YLB-03) and prepared three kinds of dosage form, analyzed by transdermal experiments and high-performance liquid phase (HPLC) content analysis. Three dosage forms (liniments, gels and ointments) of Candesartan were prepared in different concentrations (0.03%, 0.3% and 3%). Then, different proportional of transdermal promoters was added to perform transdermal experiments. The transdermal effects of three kinds of YLB-01 formulation were evaluated by the modified Franz diffusion pool. Cumulative transmittance of liniments, gels and ointments in 0.3% concentration of YLB-01 for 9 hours was measured respectively. Results: (1) The best transdermal promoter ratio of liniments, gels and ointments were 2% propylene glycol+2% azone, 4% propylene glycol, and 5% propylene glycol+2% azone. (2) 9 hours of cumulative transmittance of 0.03%, 0.3%, 3% liniments were 4.23%, 10.96% and 12.17%, respectively; 9 hours of cumulative transmittance of 0.03%, 0.3%, 3% gels were 2.29%, 4.43% and 13.57%, respectively; 9 hours of cumulative transmittance of 0.03%, 0.3%, 3% ointments were 2.08%, 1.12% and 0.73%, respectively. Conclusion: The different contents of Candesartan liniments, gels and ointments are all have some transdermal abilities, and the abilities are: liniments>gels>ointments.

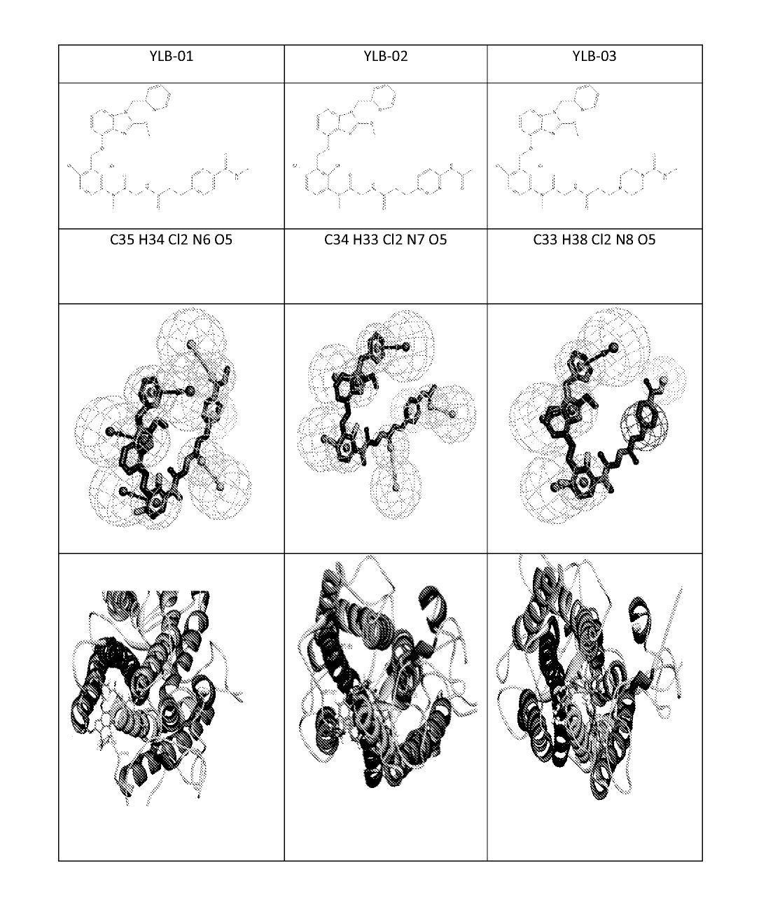

To determine the optimal dosage form of YLB-01 and best transdermal promoter, we plan to use transdermal test combined with HPLC content analysis as screening basis, research on dosage forms and transdermal promoter to achieve a stable and reliable preparation. The samples were analyzed by HPLC on an Agilent TC-C18 column (150 mm×4.6 mm, 5 microns). The mobile phases were methanol and 0.02 mol/L potassium dihydrogen phosphate buffer (66:34, pH was adjusted to 4 with phosphoric acid). Flow rate: 1 ml/min; Column temperature: 30° C. Excitation wavelength and fluorescent wavelength of fluorescence detection were 267 nm and 380 nm, respectively. Under this condition, YLB-01 retention time was 3.898 min, target compounds peak shape is good which could be effectively separated, and no impurity peak, baseline is smooth (shown as FIG. 6).

### Example 6

In order to optimize YLB-01 formulation, the liniments, gels and ointments three dosage forms of YLB-01 were prepared, and in each dosage form there were three concentrations, which were 0.03%, 0.3%, 3%, respectively. Then, the best dosage form and concentration were determined. In order to prepare liniment for YLB-01, we mixed YLB-01, ethyl alcohol, 0.1M NaOH, propylene glycol, azone and glycerol and added distilled water to full amount, and stirred to dissolve.

In order to prepare YLB-01 solid dispersions, we weighed YLB-01 and PVPK-30, with the right amount of mixed solvent (methylene chloride, methanol=1:1) heating and stirring to dissolve YLB-01, then ethanol dissolves the carrier, to be fully dissolved to clarify. Two kinds of solution was mixed and stirred to clear solution. Then, it was put into the round bottom flask, 45° C. reduced pressure distillation to dry solvents. After removing solvent to become white solid, then, it was rapidly frozen −20° C. for 2 h, and then placed in 60° C. oven to remove residual solvent, inside the dryer saved for later use.

YLB-01 gels were obtained as follows. Propylene glycol, azone, glycerin, caprylyl glycol, ethylparaben were mixed and heated to dissolve; YLB-01 was added, with full amount of water, and stirred to dissolve; carbomer 940 was added for overnight swelling, then appropriate amount of triethanolamine was added to form transparent gels.

0.3% (by weight) YLB-01: Glycerylmonostearate, stearic acid, white petrolatum, and liquid paraffin is heated to melt as oil phase. Glycerol, SDS, ethyl paraben and water were heated to 90° C. as aqueous phase. Candesartan was added into propylene glycol and PEG400, and heated until completely dissolved. The oil phase was then slowly added to the aqueous phase, and YLB-01 was added with stirring. Stirring was continued until the temperature lowered to room temperature. Then, Azone was added. Keep stirring until the solution condensate.

### Example 7

To study transdermal effect of different prescription compatibility, the 0.3% content of the liniments, gels and ointments samples were selected to be used in the first round of screening, and the results were shown in Table 9; After the first round of screening, the optimal concentration of transdermal promoter and compatibility was determined, and on this condition, the other 2 concentration (0.03%, 3%) were compared.

TABLE 9
The results of the first round of screening of
transdermal absorption promoting agent

transdermal absorption
promoting agent (%)

| Dosage form | No. | propylene glycol | Azone |
|---|---|---|---|
| Liniments | 1 | 0 | 0 |
| | 2 | 4 | 0 |
| | 3 | 0 | 4 |
| | 4 | 2 | 2 |
| Gels | 1 | 0 | 0 |
| | 2 | 2 | 0 |
| | 3 | 4 | 0 |
| Ointment | 1 | 5 | 0 |
| | 2 | 5 | 2 |
| | 3 | 5 | 4 |

The abdominal hair of mice was removed. The mice were sacrificed by spinal dislocation. The subcutaneous fat was removed, and the depilous skin of abdomen was reserved in 4° C. physiological saline. Skin was fixed between the supply room and reception room facing the supply room. The effective permeability area was 4 cm². Receiving liquid was physiological saline, and receiving chamber volume V were 25 ml. 1.0 g of the tested sample was coated evenly on the effective skin surface with the electromagnetic stirrer run at a speed of 300 r/min. To take out the receiving liquid in 1, 2, 3, 4, 5, 6, 7, 8, 9 h, 0.5 ml/h (Complementing the same quantity each time), the samples were centrifuged and the supernatant was took for HPLC analysis.

After the detection, the cumulative transmittance P (%) was calculated:

$$P ( \% ) = \frac{Cn \times V + {\sum }_{i = 1}^{n - 1} Ci \times 0.5}{m} \times 100 \%$$

V: the volume of receiving liquid in receiving room; Ci: drug concentration in receiving liquid between the time to last time of the i time; Cn: drug concentration in the receiving liquid when the n time taking the sample; M: drug content in the sample.

To detect the content of YLB-01 of receiving liquid by HPLC, the optimal dosage forms and the best proportion of transdermal absorption promoting agent were selected. 1.0 g/L YLB-01 standard stock solution was prepared as follows. 10.0 mg of Candesartan was added into methanol solution, once melt it and diluted to 10 ml scale, and mixed well. The concentrations of 80 mg/L, 20 mg/L, 10 mg/L, 5 mg/L, 1 mg/L of YLB-01 methanol standard solution were prepared from standard stock solution.

The Standard Curve was Obtained as Follows.

950 μl of normal saline, was added into 50 μl series of different concentration of standard solution to acquire the standard samples which included 4000, 1000, 500, 250, 50 m/l YLB-01. 20 μl each time was injected, and the map recorded, the concentration (μg/l) was as the abscissa, peak area was as the verticalcoordinates, regression equation was Y=12224 x+437621, r²=0.9998, and the linear range was 50 m/l˜4000 μg/l.

According to the results of screening of the permeation enhancers, liniments, gels and ointments which contained 0.03%, 0.3% and 3% YLB-01 respectively were detected the cumulative permeation rate for 9 hours. The results showed that the transdermal effects were liniments>gels>ointments. The middle and high concentrations of liniments could get good transdermal effects, and high concentration of gels indicated high permeation rate. However, three concentrations of ointments didn't show good transdermal effects. In addition, the permeation rate declined with the content of YLB-01 increasing, which implied PEG400 may hinder the transdemal absorption and the form of YLB-01 dispersing in the cream base may affect the transdemal effect (Table 10).

TABLE 10
Transdermal effects of three dosage forms of YLB-01

| | | | | Cumulative |
|---|---|---|---|---|
| | Content of | Penetration Enhancers | | Penetration |
| Dosage | YLB-01 | (%) | | Rate of 9 Hours |
| Forms | (%) | propylene glycol | azone | (%) |
| liniments | 0.03 | 2 | 2 | 4.23 |
| | 0.3 | 2 | 2 | 10.96 |
| | 3 | 2 | 2 | 12.17 |
| gels | 0.03 | 4 | 0 | 2.29 |
| | 0.3 | 4 | 0 | 4.43 |
| | 3 | 4 | 0 | 13.57 |
| ointments | 0.03 | 5 | 2 | 2.08 |
| | 0.3 | 5 | 2 | 1.12 |
| | 3 | 5 | 2 | 0.73 |

### REFERENCES

1. Calixto J B, Cabrini D A, Ferreira J, Campos M M. Kinins in pain and inflammation. Pain 2000; 87:1-5.
2. Heitsch H. Bradykinin B2 receptor as a potential therapeutic target. Drug News Perspect 2000; 13:213-225.
3. Sasaki S, Hozumi Y, Kondo S. Influence of prostaglandin F2a and its analogues on hair regrowth and follicular melanogenesis in a murine model. Exp Dermatol. 2005; 14:323-328.
4. Alhenc-Gelas F, Bouby0000 N, Richer C, Potier L, Roussel R, Mane M. Kinins as therapeutic agents in cardiovascular and renal diseases. Curr Pharm Des. 2011; 17(25):2654-2662.
5. Johnstone M A, Albert D M. Prostaglandin-induced hair growth. Surv Ophthalmol. 2002; 47 Suppl 1:S185-202.
6. Gether U, Kobilka B K. G Protein-coupled Receptors. J Biol Chem, 1998; 273(29): 17979-17982.
7. Marvin C. Gershengorn and Roman Osman, Minireview: Insights into G Protein-Coupled Receptor Function Using Molecular Models. Endocrinology, 2001; 142: 2-10.
8. Ha S N, Hey P J, Ransom R W, Harrell C Jr, et al, Binding modes of dihydroquinoxalinones in a homology model of bradykinin receptor 1. Biochemical and Biophysical Research Communications. 2005; 331:159-166.
9. Su D S, Markowitz M K, DiPrado R M, Murphy K L, Harrell C M, O'Malley S S, Ransom R W, Chang R S, Ha S, Hess F J, Pettibone D J, Mason G S, Boyce S, Fredinger R M, Bock M G. Discovery of a potent, non-peptide Bradykinin B1 receptor antagonist, J. Am. Chem. Soc. 2003; 125:7516-7517.
10. Underwood D J, Strader C D, Rivero R, Structural model of antagonist and agonist binding to the angiotensin II, AT, subtype, G protein coupled receptor, Chem Biol. 1994; 1:211-221.
11. Gieldon A, Lopez J J, Glaubitz C, Schwalbe H. Theoretical Study of the Human Bradykinin Bradykinin B2Receptor Complex. ChemBio Chem, 2008; 9:2487-2497.
12. Hu Q, Song H, Yang L, et al. The Preparation and Clinical Application of Benzene Liquid Liniment, The Chinese and Western Medical Journal, 2011; 9(6): 63-64.
13. Chen H, Wong T, Chang X, et al. Preparation of Ibuprofen Microemulsion and Its Transdermal Absorption. Chinese Pharmaceutical Journal, 2004, 39(1): 43-45.
14. Du S, Gu R, Chen H. The Screening and Stability Study of the Prescription of Cream Base. Chongqing Medical Journal, 2002, 31(7): 632-633.
15. Lin G T, Sheng H G, Zhang C. Study on vitro Transdermal Absorption of FufangChansuZhengtong Cataplasm, Chinese Journal of Experimental Traditional Medical Formulae, 2011; 17(9): 1-1.
16. Wang D, Han G Z, Chen Z J. Effects of Penetration Enhancers on in vitro Percutaneous Permeation of Tea Polyphenols Liniment. Chinese Journal of New Drugs, 2010; (13): 1171-1174.
17. Nelson A M, Loy D E, Lawson J A, Katseff A S, Fitzgerald G A, Garza L A. Prostaglandin D2 inhibits wound-induced hair follicle neogenesis through the receptor, Gpr44. J Invest Dermatol. 2013; 133(4):881-9.
18. Garza L A, Liu Y, Yang Z, Alagesan B, Lawson J A, Norberg S M, Loy D E, Zhao T, Blatt H B, Stanton D C, Carrasco L, Ahluwalia G, Fischer S M, FitzGerald G A, Cotsarelis G. Prostaglandin D2 inhibits hair growth and is elevated in bald scalp of men with androgenetic alopecia. Sci Transl Med. 2012; 4(126): 126-134.
19. Colombe L, Michelet J F, Bernard B A. Prostanoid receptors in anagen human hair follicles. Exp Dermatol. 2008; 17(1):63-72.
20. Böhm M, Schiller M, Luger T A. Non-pigmentary actions of alpha-melanocyte-stimulating hormone-lessons from the cutaneous melanocortin system. Cell Mol Biol (Noisy-le-grand). 2006; 52(2):61-8.
21. Malkinson F D, Geng L, Hanson W R. Prostaglandins protect against murine hair injury produced by ionizing radiation or doxorubicin. J Invest Dermatol. 1993; 101(1 Suppl):1355-1375.

What is claimed is:

1. A topical composition comprising at least a bradykinin (BK) B2 receptor agonist as an active ingredient.

2. The topical composition according to claim 1, wherein said active ingredient has a concentration of from about 0.01% to about 10% by weight.

3. The topical composition according to claim 1, wherein said active ingredient has a concentration of from about 0.03% to about 3% by weight.

4. The topical composition according to claim 3, wherein said active ingredient has a concentration of about 0.03%.

5. The topical composition according to claim 3, wherein said active ingredient has a concentration of about 0.3%.

6. The topical composition according to claim 3, wherein said active ingredient has a concentration of about 3%.

7. The topical composition according to claim 1, wherein said active ingredient is a BK B2 receptor agonist selected from the group consisting of YLB-02, YLB-02 and YLB-03.

8. The topical composition according to claim 1, wherein said active ingredient has a concentration of from 10 nM to 2000 μM

9. The topical composition according to claim 1, wherein said active ingredient is a non-peptide compound.

10. The topical composition according to claim 1, wherein said active ingredient, YLB-01, is 4-(3-2-((2,4-dichloro-3-((2-methoxy-1-(pyridine-2ylmethyl)-1H-benzo[d]imidazol-4-yloxy)methyl)phenyl)(methyl)amino-2-oxoethylamino)-3-oxopropyl)-N-methylbenzamid.

11. The topical composition according to claim 1, wherein said active ingredient, YLB-02, is 3-(6-acetamidopyridin-3-yl)-N-(2-((2,4-dichloro-3-((2-methoxy-1-(pyridine-2-ylmethyl)-1H-benzo[d]imidazol-4yloxy)methyl)phenyl)(methyl)amino-2-oxoethyl) propanamide.

12. The topical composition according to claim 1, wherein said active ingredient, YLB-03, is 4-{2-[({[2,4-Dichloro-3-(2-methoxy-1-pyridin-2-ylmethyl-1H-benzoimidazol-4-yloxymethyl)-phenyl]-methyl-carbamoyl}-methyl)-carbamoyl]-ethyl}-piperazine-1-carboxylic acid methylamide.

13. The topical composition according to any one of claims 1-12, further comprising at least a pharmaceutical carrier selected from the group consisting of transdermal permeation enhancer, transdermal absorption promoting agent, water, solvent, preservative, surfactant, and a pH balancer.

14. The topical composition according to claim 13, wherein said transdermal permeation enhancer is propylene glycol, Azone, or a combination thereof.

15. The topical composition according to claim 14, wherein said propylene glycol or Azone has a concentration of 2% by weight.

16. The topical composition according to claim 13, comprising alcohol and/or a PBS solution.

17. The topical composition according to claim 13, wherein said PBS solution has a pH at from about 6.5 to about 7.8.

18. The topical composition according to any one of claims 1-17, wherein said composition is in the form of a gel, liniment, cream or ointment.

19. Use of a topical composition according to any one of claims 1-18 in the preparation of a medicament for reducing or delaying hair loss.

20. The use according to claim 19, wherein said hair loss is caused by androgenetic alopecia, or seborrheic alopecia.

21. Use of a topical composition according to any one of claims 1-18 in the preparation of a medicament for increasing or promoting hair growth.

22. The use according to claim 21, wherein said hair growth is eye brow or eye lash growth.

23. The use according to claim 19 or 21, wherein said hair loss or growth is related to PG E/F2a release.

24. A method of reducing or delaying hair loss in a subject comprising administering a topical composition according to any one of claims 1-18 to said subject in an amount effective to reduce or delay hair loss.

25. The method according to claim 24, wherein said hair loss is caused by androgenetic alopecia, or seborrheic alopecia.

26. A method of increasing or promoting hair growth in a subject, comprising administering a topical composition according to any one of claims 1-18 to said subject in an amount effective to increase or promote hair growth.

27. The method according to claim 26, wherein said hair growth is eye brow or eye lash growth.

28. The method according to claim 24 or 26, wherein said hair loss or growth is related to PG E/F2a release.

29. The method according to any one of claims 24-28, wherein the daily therapeutic dose of the active ingredient administered is from about 0.01 mg to about 500 mg.

30. The method according to claim 29, wherein the daily therapeutic dose of the active ingredient administered is from about 0.1 mg to about 300 mg.

31. The method according to claim 30, wherein the daily therapeutic dose of the active ingredient administered is from about 1 mg to about 250 mg.

32. A kit comprising said topical composition according to any one of claims 1-18.

33. A computer modeling structure identifying a series of BK B2 receptor agonist binding sites of within BK B2 receptor.

34. The computer modeling structure according to claim 33, wherein said binding sites are presented in 2-dimentions or 3-dimentions.

35. The computer modeling structure according to claim 33, wherein said binding sites are identified using molecular modeling.

36. The computer modeling structure according to claim 35, wherein said molecular modeling is based on one or more BK B2 receptor agonists selected from the group consisting of YLB-01, YLB-02, and YLB-03.

37. The computer modeling structure according to claim 36, wherein residues found at YLB-01 binding pocket border contain one or more selected from the group consisting of G205, I219, Y322, E204, T314, W5, and R297.

38. The computer modeling structure according to claim 36, wherein residues found at YLB-02 binding pocket border contain one or more selected from the group consisting of ARG338, TYR332, GLU93, and ARG167.

39. The computer modeling structure according to claim 36, wherein residues found at YLB-03 binding pocket border contain one or more selected from the group consisting of VAL91, GLN352, ARG338, and TYR347.

40. The computer modeling structure according to claim 36, wherein YLB-01 binding site contains one or more residues selected from the group consisting of T95, R55, and R338.

41. The computer modeling structure according to claim 36, wherein, YLB-02 binding site contains one or more residues selected from the group consisting of E93, Y332, and R338.

42. The computer modeling structure according to claim 36, wherein YLB-03 binding site contains one or more residues selected from the group consisting of V91, R338, Y347, and Q352.
